(19) United States
(12) Patent Application Publication
(10) Pub. No.: US 2007/0106357 A1
(43) Pub. Date: May 10, 2007

(54) Intravascular Electronics Carrier Electrode for a Transvascular Tissue Stimulation System

(76) Inventors: Stephen Denker, Mequon, WI (US); Arthur J. Beutler, Greendale, WI (US); Cherik Bulkes, Sussex, WI (US)

Correspondence Address: QUARLES & BRADY LLP, 411 E. WISCONSIN AVENUE, SUITE 2040, MILWAUKEE, WI, 53202-4497, US

(21) Appl. No.: 11/553,264

(22) Filed: Oct. 26, 2006

Related U.S. Application Data
(60) Provisional application No. 60/734,019, filed on Nov. 4, 2005.

(51) Int. Cl. A61N 1/00

(52) U.S. Cl. 607/116; 607/119

(57) ABSTRACT

An intravascular mesh type electrode carrier eliminates material and mechanical transitions between an electrical lead and an electrode by interweaving the conductor of the lead into the carrier mesh. The mesh material of the electrode carrier can be electrically conductive so that the entire carrier functions as the electrode. Alternatively, the mesh material may either be non-conductive or have an outer non-conductive coating, in which cases only the exposed section of the first conductor acts as an electrode. With a non-conductive mesh material, a second electrode lead can be woven through the mesh of the electrode carrier to provide a second electrode.

### CROSS-REFERENCE TO RELATED APPLICATIONS

This application claims benefit of U.S. Provisional Patent Application No. 60/734,019 filed Nov. 4, 2005.

### STATEMENT REGARDING FEDERALLY SPONSORED RESEARCH OR DEVELOPMENT

Not Applicable

### BACKGROUND OF THE INVENTION

1. Field of the Invention

The present invention relates implantable electronic medical devices, such as cardiac pacemakers and defibrillators for example, for stimulating tissue of animal for therapeutic purposes; and more particularly to electrode assemblies for such devices.

2. Description of the Related Art

A remedy for a patient with certain physiological ailments is to implant a stimulation device that applies an electrical pulse to an organ or part of an organ which is affiliated with the ailment. The stimulation device includes an electronic pulse generator from which electrical leads extend to electrodes in contact with parts of the organ, which when electrically stimulated provide therapy to the patient.

U.S. Pat. No. 6,907,285 discloses an improved apparatus, that is implanted in the blood vascular of an animal for physiological stimulation of cardiac tissue. That apparatus is formed by a transvascular platform which includes at least one intravascular electronics-electrode carrier on which is mounted a wireless radio frequency (RF) receiver and an electronic capsule containing stimulation circuitry. The stimulation circuitry receives a radio frequency signal from an extracorporeal power supply and derives an electrical voltage from the energy of that signal. The voltage is applied in the form of suitable electrical waveforms to electrodes, thereby stimulating the tissue.

A tubular, mesh stent is commonly used to enlarge a constricted blood vessel. The stent has a collapsed state in which its diameter is minimized to enable insertion on a catheter through the blood vessels to the region of constriction. At that location, the stent is released and enlarges diametrically so that is outer circumference expands the blood vessel. The mesh stents are formed of stainless steel, Nitinol or similar shape-memory material. Similar stents have been proposed for securing stimulation electrodes in blood vessels.

A challenge in designing an intravascular electronics-electrode carrier is to provide a means for reliably connecting an electrode to a carrier, such as a stent. A common technique connected the electrode by a material/mechanical transition, such as a helical spring coil or a welded joint. This type of transition provided a point of potential failure of the apparatus due to mechanical fatigue and posed a limitation on the flexibility of the electrode/carrier assembly.

### SUMMARY OF THE INVENTION

In an implanted medical device for stimulation of body tissue, a material and mechanical transition between an electrical lead and an electrode carrier is avoided by incorporating the conductor of the lead into the carrier.

An electrode assembly, for implantation into an animal to stimulate tissue inside the animal, comprises an electrode carrier and first electrode lead. The electrode carrier of a mesh material is adapted to contact the tissue. The first electrode lead for a stimulation signal has a first conductor encased in electrical insulation. A portion of the first electrode lead is woven through the mesh of the electrode carrier and a section of the first conductor in that first portion is devoid of the first electrical insulation, thereby forming an electrode.

The mesh material of the electrode carrier can be electrically conductive so that the entire carrier functions as the electrode. Alternatively the mesh material may either be non-conductive or have an outer non-conductive sheath, in which cases only the exposed section of the first conductor acts as an electrode.

In another aspect of the invention, a second electrode lead also is woven through the mesh of the electrode carrier. The second electrode includes a second conductor with a section that is devoid of electrical insulation, thereby forming another electrode. In this version the exterior surfaces of the electrode carrier are electrically non-conductive and the exposed sections of the first and second conductors are electrically separated from each other.

Several arrangements of one or two electrode carriers of this type are utilized in implanted medical devices described herein for stimulating the tissue of an animal.

### BRIEF DESCRIPTION OF DRAWINGS

FIG. 1 depicts intracorporeal and extracorporeal components of a wireless transvascular platform for tissue stimulation;

FIG. 2 is a schematic diagram of a first embodiment of an intravascular electrode carrier that is an intracorporeal component of the transvascular platform;

FIG. 3 is an isometric view of a portion of an electrical lead for the intravascular electrode carrier;

FIG. 4 is a schematic diagram of a second embodiment of an intravascular electrode carrier to which a pair of electrical lead connect;

FIG. 5 is a schematic diagram of a third embodiment of an intravascular electrode carrier connected to a single electrical lead connect having two conductors;

FIG. 6 shows an electrode configuration of the medical apparatus for left atrial/ventricular pacing and sensing;

FIG. 7 illustrates a electrode configuration for defibrillation and left atrial and ventricular pacing and sensing; and

FIG. 8 is an electrode carrier configuration for atrial fibrillation treatment.

### DETAILED DESCRIPTION OF THE INVENTION

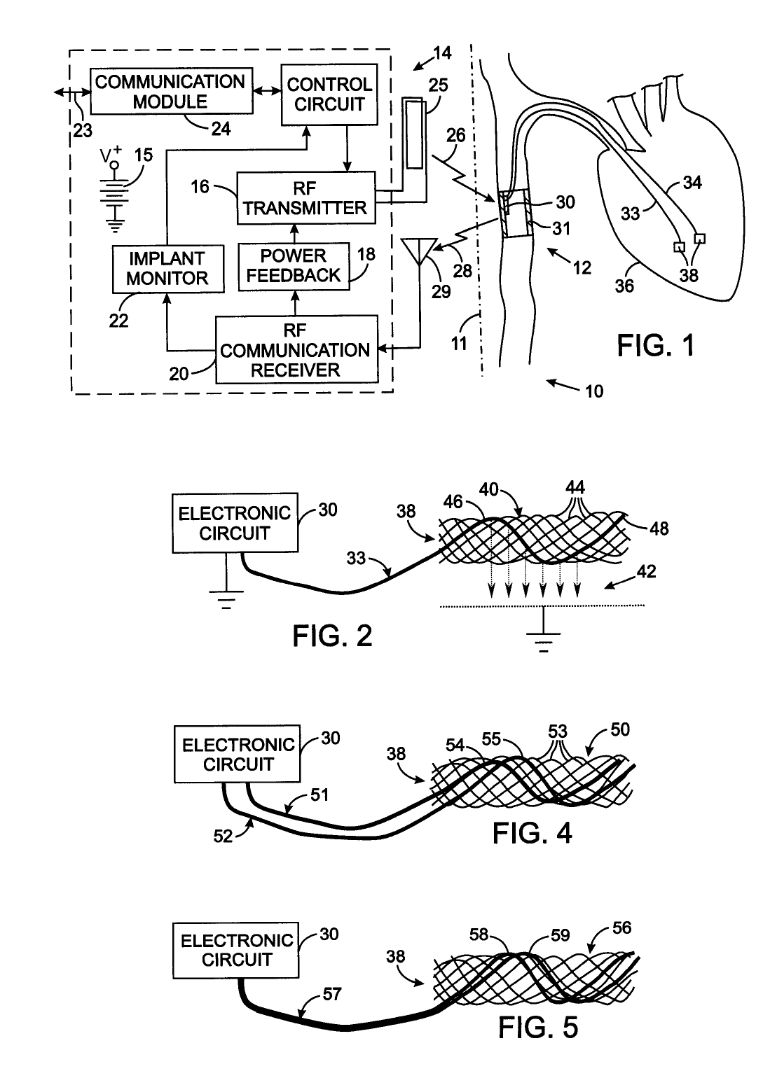

With initial reference to FIG. 1, a wireless transvascular platform 10 for tissue stimulation includes a medical device 12 implanted inside the body 11 of an animal and an external power supply 14. The medical device stores energy received via radio frequency signal from the external power supply 14 and used that energy to power an electronic circuit 30 mounted on an electronic carrier 31.

The external power supply 14 includes a battery 15, a radio frequency (RF) power transmitter 16, a power feedback module 18, an RF communication receiver 20, and an implant monitor 22. In addition, an optional communication module 24 may be provided to exchange data and commands via communication link 23 with other apparatus (not shown), such as a programming computer or patient monitor. The communication link 23 may be a wireless link such as a radio frequency signal or a cellular telephone call.

The battery 15 is rechargeable allowing for patient mobility with periodic recharge cycles. Depending upon the type and size of the battery, the time between recharge cycles may be days, months or years. Power transmitter 16 and a first antenna 25 periodically transmit a first radio frequency signal 26 that is pulse width modulated (PWM) to convey varying amounts of energy to the medical device 12. The medical device 12 uses that energy to charge an electrical storage device in the electronic circuit 30. The charge of the storage device is monitored and the electronic circuit 30 sends data indicating its power needs via a second radio frequency signal 28. The second radio frequency signal is received at the external power supply 14 by a second antenna 29 and the RF communication receiver 20. The power feedback module 18 is part of closed loop system that receives the medical device's power needs data and responds by controlling the duty cycle of the first radio frequency signal 26 to ensure that the medical device 12 has a sufficient amount of electrical power.

The implant monitor 22 receives other data, such as physiological conditions of the animal, status of the medical device and trending logs, for example, that have been collected by the implanted electronic circuit 30 and sent via the second radio frequency signal 28. This data is provided to the communication module 24 so that medical personnel can review the data or be alerted when a particular condition exists.

Referring still to FIG. 1, the implanted medical device 12 includes the electronic circuit 30 mentioned above which includes an RF transceiver and a tissue stimulation circuit, similar to that used in conventional pacemakers and defibrillators. That electronic circuit 30 is located in a large blood vessel 32, such as the inferior vena cava (IVC), for example. One or more, electrical leads 33 and 34 extend from the electronic circuit through the animal's blood vasculature to locations in the heart 36 where pacing and sensing are desired. Each lead has an electrical conductor enclosed in an electrically insulating outer layer. The electrical leads 33 and 34 terminate at electrode assemblies 38 at those locations.

FIG. 2 illustrates the details of a first embodiment of the electrode assembly 38 comprising an electrode carrier 40 to which an electrode lead 33 from the electronic circuit 30 connects. A mesh-type electrode carrier 40 can be employed which is similar to stents commonly used to enlarge constricted blood vessels. That type of carrier comprises a plurality of wires formed to have a memory defining a tubular shape or envelope. Those wires may be heat-treated platinum, Nitinol, a Nitinol alloy wire, stainless steel, plastic wires or other materials. Plastic or substantially nonmetallic wires may be loaded with a radiopaque substance which provides visibility with conventional fluoroscopy. A catheter assembly is used to implant the components of the medical device 12 in the animal's circulatory system. The electrode carrier 40 has a shape memory so that it normally assumes an expanded tubular configuration when unconfined, but is capable of assuming a collapsed configuration when disposed and confined within a catheter assembly. In that collapsed state, the electrode carrier 40 has a relatively small diameter enabling it to pass freely through the vasculature of an animal. After being properly positioned in the desired blood vessel, the electrode carrier is released from the catheter assembly and expands to engage the blood vessel wall thereby becoming anchored in place.

The electrode lead 33 from the electronic circuit 30 extends to the electrode carrier 40 where a bare conductor 46 of the lead engages the electrode carrier. A conduction path 42 through tissue of the animal completes an electrical circuit back to the electronic circuit 30. One form of a mesh-type electrode carrier 40 has a plurality of helical, metal strands 44 that are interwoven to form a tube. In one embodiment, the strands 44 are electrically conductive and contact the bare conductor 46 portion of the electrode lead 33 which is helically woven with the strands of the carrier. Thus, the entire electrode carrier 40 functions as the electrode. Alternatively, the strands 44 are formed of non-conductive material or are insulated by a non-conductive surface coating, in which cases only the bare conductor 46 portions of the electrode lead functions as the electrode.

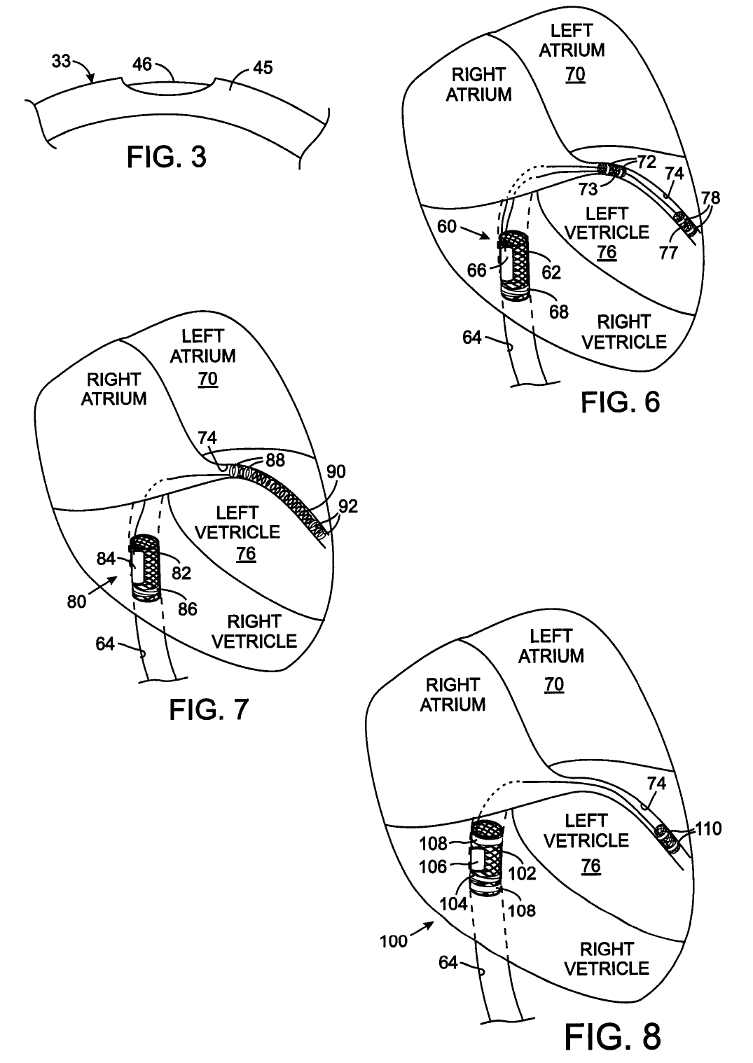

As a further variation, the conductor 46 can be exposed only at remote tip of the electrode lead, thereby creating a focused point of electrical contact with tissue of the animal. Another variation is shown in FIG. 3, in which a portion of the outer insulating layer 45 facing outward from the carrier is removed from the electrode lead 33 to expose a small section of the inner electrical conductor 46, thus forming an electrode at that region of the lead. Additional portions of the outer insulating layer 45 can be removed to form more electrodes along the lead 33. In all those alternative, the bare conductor 46 does not have to extend the full length of the electrode carrier.

The materials that form the inner electrical conductor 46, and the electrode carrier 40 must possess certain characteristics, such as fatigue resistance to flexing (especially for components to be implanted in or near the apex of heart or the ventricles) and high electrical conductivity. The components such as the outer insulating layer 45 of the electrode lead 33, and the electrode carrier 40 must be compatible with the tissue in which they will be implanted. Those components must exhibit resistance to adverse biological reactions and to formation of insulating oxides. Examples of suitable materials include stainless steel and alloys containing silver, nickel and chromium.

In a second embodiment of the electrode assembly 38 shown in FIG. 4, a pair of electrode leads 51 and 52 extend from the stimulator electronic circuit 30 to an electrode carrier 50 that has the same structure as electrode carrier 40 previously described. However, the stands 53 of the electrode carrier 50 do not have electrically conductive surfaces, i.e. the strands either are formed of non-conductive material or have a non-conductive surface coating. Bare conductors 54 and 55 portions of the two electrode leads 51 and 52, respectively, are interwoven into the stands of the electrode carrier 50. The bare conductors 54 and 55 are arranged so that they do not contact each other. Alternatively, only a small section of each electrode leads 51 and 52 in the electrode carrier 50 have an exposed conductor with those portions being spaced apart longitudinally along the electrode carrier 50. An electrical circuit is completed between the bare conductors 54 and 55 by the adjacent animal tissue, thereby providing localized stimulation or sensing.

With reference to FIG. 5, a third embodiment of the electrode assembly 38 comprises an mesh-type electrode carrier 56 that has electrically non-conducting exterior surfaces similar the electrode carrier 50. A single electrode lead 57 extends from the stimulator electronic circuit 30 and has two electrically insulated conductors 58 and 59 encased in an insulating outer sheath. The two electrically insulated conductors 58 and 59 emerge from the outer sheath at the electrode carrier 50 and have their individual outer insulations removed from portions that are woven through the mesh of the electrode carrier 56. The exposed portions of each conductor are electrically separated from each other.

The design of a medical device that provides a reliable connection of an electrode to the conductor of the lead and to the electrode carrier requires configurations that are customized to the specific application of that device in the animal's body. FIGS. 6-8 show three exemplary configurations for such specific applications. For ease of illustration the electrodes in these figures are shown as being ring-shaped.

FIG. 6 shows an implanted medical device 60 using the new transvascular framework for cardiac pacing. An electronics carrier 62, having an expandable stent-like mesh structure, is deployed in the inferior vena cava 64 and holds the RF receiving coil 68 and an electronic capsule 66 containing the electronic circuit 30. For pacing the left atrium 70, a first pair of positive and negative electrodes 72 are mounted on a first electrode carrier 73 placed at the proximal end of coronary sinus 74. To pace the left ventricle 76, a second pair of positive and negative electrodes 78 are mounted on a second electrode carrier 77 that is placed at the distal end of coronary sinus 74. Each electrode in pairs 72 and 78 is connected to the electronic capsule 66 by a separate conductor in an electrical lead.

Both the positive and negative pacing electrodes are on the same electrode carrier 73 and 77 spaced approximately one centimeter apart, for example. Therefore, the carrier cannot have any conductive material bridging the two electrodes. Alternatively, separate electrode carriers could be used for each electrode. FIG. 6 illustrates a configuration for bipolar pacing, in which a pair of ring-shaped electrodes are provided for each heart chamber, with each ring having a small (e.g. 1.0 mm) diameter. For other forms of pacing, a single ring or a curved plate may be placed adjacent the left ventricle 76 and another similar electrode near the left atrium 70.

FIG. 7 depicts an intravascular electronics-electrode carrier configuration 80 for defibrillation and pacing. An electronics carrier 82, similar to the one in FIG. 6, holds an electronics capsule 84 and a receiving coil 86. A first pair of electrodes 88 are mounted on an electrode carrier 90 placed at the proximal end of coronary sinus 74. This electrode carrier 90 is a continuous elongated wire strand that extends through the coronary sinus 74 for a distance of 10.0 cm, for example, to a distal end at a point adjacent the left ventricle 76. The first pair of electrodes 88 at the proximate end of the carrier sense cardiac activity adjacent the left atrium 70, and a second pair of electrodes 92 at the distal end sense cardiac activity adjacent the left ventricle 76. During defibrillation, the entire electrode carrier 90 becomes an anode and a metallic housing of the electronics capsule 84 or the receiving coil 86 functions as a cathode. The electrode-carrier 90 in this situation is similar to coils on leads of a traditional implanted cardiac defibrillator (ICD). Alternatively the polarity of the defibrillation pulses applied to the capsule 84 and the electrode carrier 90 can be reversed, as well as being biphasic.

FIG. 8 illustrates an electronics-electrode carrier configuration 100 for treatment of atrial fibrillation. Here, a combined electronics-electrode carrier 102 is expanded to become embedded in the wall of the inferior vena cava 64 adjacent to the vagal nerve. The electronics-electrode carrier 102 holds an RF receiver coil 104 and an electronic capsule 106, that is connected to two stimulation electrodes 108 extending circumferentially around the carrier in contact with the wall of the blood vessel. Electrical pulses are applied across those electrodes 108 to stimulate the vagal nerve. Electrical leads run from the electronic capsule 106 to a remote pair of electrodes 110 located in the coronary sinus 74 to sense cardiac activity at that site. While the vagal nerve is being stimulated by the stimulation electrodes 108 and activity of the left ventricle can be sensed using the remote pair of electrodes 110. Alternatively, the vagal nerve stimulation can be performed at the site in the coronary sinus 74 or other cardiac vasculature sites.

It should be noted that in preferred configurations in FIGS. 6-8, each electrode can be formed by a single helical strand of the mesh stent that forms the respective electrode carrier with an electrical effect very similar to that of a ring-shaped electrode extending around the carrier. In this case, a remaining part of the electrode carrier is insulated. Using a carrier strand in this manner eliminates potential problems associated with welded joints at the electrode to electrode carrier interface. In another version, a plurality of contact electrodes are mounted on, but insulated from a metal carrier. Here, the circuitry in the electronic circuit is able to select different electrodes to act as the anode and cathode for stimulation. This redundancy provides improved reliability of the stimulation delivery system as it allows selection of another electrode, if poor conductivity is detected at a particular electrode. Alternatively, several combinations of positive and negative electrode pairs can be sequentially tested by the electronic circuit and the pair that provides the best response can be chosen for the stimulation. In yet another embodiment, each electrode is mounted on a separate electrode carrier. Further, each electrode may be chosen by the electronic circuit to be negative or positive with respect to the metal housing of the electronic capsule.

It should be further noted that one of the electrodes or the electronic capsule may be located in any other suitable vessel, such as a basilic vein for example, instead of being located at inferior vena cava.

The foregoing description was primarily directed to a preferred embodiment of the invention. Although some attention was given to various alternatives within the scope of the invention, it is anticipated that one skilled in the art will likely realize additional alternatives that are now apparent from disclosure of embodiments of the invention. Accordingly, the scope of the invention should be determined from the following claims and not limited by the above disclosure.

1. An electrode assembly for implantation to stimulate tissue inside an animal, said electrode assembly comprising:
an electrode carrier of a mesh material adapted for contacting the tissue; and
a first electrode lead having a first conductor and electrical insulation around the first conductor, wherein a first portion of the first electrode lead is woven through the mesh of the electrode carrier and a first section of the first conductor in that first portion is devoid of the electrical insulation.

2. The electrode assembly as recited in claim 1 wherein the electrode carrier is formed strands of material that are interwoven.

3. The electrode assembly as recited in claim 1 wherein the mesh material of the electrode carrier is electrically conductive and the first section of the first conductor is woven through the electrode carrier.

4. The electrode assembly as recited in claim 1 wherein the electrode carrier has an electrically conductive surface in contact with the first conductor and the tissue.

5. The electrode assembly as recited in claim 1 wherein the electrode carrier has an electrically nonconductive surface.

6. The electrode assembly as recited in claim 1 wherein first conductor has a second section that is devoid of the electrical insulation.

7. The electrode assembly as recited in claim 1 wherein the first electrode lead further comprises a second conductor enclosed in additional electrical insulation, wherein a second section of the second conductor in the first portion is devoid of the additional electrical insulation.

8. The electrode assembly as recited in claim 1 further comprising a second electrode lead having a second conductor and additional electrical insulation around the second conductor, wherein a second portion of the second electrode lead is woven through the mesh of the electrode carrier and a second section of the second conductor in that second portion is devoid of the additional electrical insulation.

9. An electrode assembly for stimulating tissue inside an animal, said electrode assembly comprising:
a first electrode carrier that is in contact with tissue in a first blood vessel of the animal;
a first electrode lead having a first conductor for a stimulation signal and first electrical insulation around the first conductor, wherein a first portion of the first electrode lead is woven through the first electrode carrier and a first section of the first conductor in that first portion is devoid of the first electrical insulation;
a second electrode carrier that is in contact with tissue in the first blood vessel; and
a second electrode lead having a second conductor and second electrical insulation around the second conductor, wherein a second portion of the second electrode lead is woven through the second electrode carrier and a second section of the second conductor in that second portion is devoid of the second electrical insulation.

10. The electrode assembly as recited in claim 9 wherein the first electrode carrier and the second electrode carrier are formed of a mesh material.

11. The electrode assembly as recited in claim 9 wherein the first electrode lead and the second electrode lead are connected to an electronic circuit implanted a second blood vessel.

12. The electrode assembly as recited in claim 11 wherein the first blood vessel is the coronary sinus and the second blood vessel is the inferior vena cava.

13. The electrode assembly as recited in claim 9 wherein the first and second electrode carriers are electrically conductive.

14. The electrode assembly as recited in claim 9 wherein the first and second electrode carriers have electrically nonconductive exterior surfaces.

15. The electrode assembly as recited in claim 9 wherein the first electrode carrier has an electrically conductive surface in contact with the first conductor and the tissue, and the second electrode carrier has another electrically conductive surface in contact with the second conductor.

16. An electrode assembly for stimulating tissue inside an animal, said electrode assembly comprising:
a first electrode carrier formed of a mesh material that is in contact with tissue of a first blood vessel in the animal;
a first electrode lead having a first conductor for a stimulation signal and electrical insulation around the first conductor, wherein a first portion of the first electrode lead is woven through the mesh of the first electrode carrier and a first section of the first conductor in that first portion is devoid of the electrical insulation; and
a second electrode carrier in a second blood vessel and having an electrode in contact with tissue of the animal.

17. The electrode assembly as recited in claim 16 wherein the second electrode carrier has an electronic circuit mounted therein and the first electrode lead and the electrode are connected to the electronic circuit.

18. The electrode assembly as recited in claim 16 wherein the first blood vessel is the coronary sinus and the second blood vessel is the inferior vena cava.

19. The electrode assembly as recited in claim 16 wherein the electrode carrier has an electrically conductive surface in contact with the first conductor and the tissue.

20. The electrode assembly as recited in claim 16 wherein the electrode carrier has an electrically nonconductive surface.

21. The electrode assembly as recited in claim 16 further comprising:
a third electrode carrier formed of a mesh material that is in contact with tissue in a blood vessel of the animal; and
a second electrode lead having a second conductor and additional electrical insulation around the second conductor, wherein a second portion of the second electrode lead is woven through the mesh of the third electrode carrier and a second section of the second conductor in that second portion is devoid of the additional electrical insulation.
